(19) United States
(12) Patent Application Publication
(10) Pub. No.: US 2024/0115342 A1
(43) Pub. Date: Apr. 11, 2024

(54) NEUROMONITORING SUPPLY KIT

(71) Applicant: Neuroplex Industries, Inc., Warwick, RI (US)

(72) Inventors: Helen Karimi, Boston, MA (US); Robert A. Beer, II, Warwick, RI (US)

(73) Assignee: Neuroplex Industries, Inc., Warwick, RI (US)

(21) Appl. No.: 18/481,316

(22) Filed: Oct. 5, 2023

Related U.S. Application Data
(60) Provisional application No. 63/378,557, filed on Oct. 6, 2022.

(51) Int. Cl. A61B 50/30

(52) U.S. Cl. CPC A61B 50/3001; A61B 2050/005; A61B 2050/3008

(57) ABSTRACT

The invention is generally directed to the novel and unique medical supply kit that is used for housing medical supplies for the rendering of medical care, such as neuromonitoring. The supply kit is also configured for receiving and containing used needles until they can be disposed of properly at the end of the surgical procedure. Also, the supply kit is reconfigurable to provide a device stand, such as for supporting a laptop, and the like. Further, the supply kit may be provided in different embodiment configurations to provide flexibility in implementation of the present invention, namely the configuration for converting the device into a needle collection vessel. The supply kit is inexpensive to manufacture, compact, easy to transport and store, and is easy to use. Therefore, the present invention provides a new and novel medical supply kit that is far superior to known kits.

### CROSS REFERENCE TO RELATED APPLICATION

This application is related to and claims priority from earlier filed US Provisional Patent Application No. 63/378,557, filed Oct. 6, 2022, the entirety thereof is incorporated herein by reference.

### BACKGROUND OF THE INVENTION

The invention related generally to medical supply kits. More specifically, the present invention relates to supply kits that are well-suited for caregivers in the field of neuromonitoring.

The supply kit of the present invention relates to intraoperative neuromonitoring, which is a supplemental health care service in which a clinician, with oversight of qualified and credentialed practitioners, performs continuous and competent monitoring of the central nervous system comprised of the brain, spinal cord, and nerves, and associated neurological structures at risk of compromise during surgical procedures.

The role of the neuromonitoring clinician is to detect anomalies in neuro-electrophysiological data and/or spectra, and communicate to the surgical care team in real-time to prevent, alter, or reduce a compromise to the nervous system and/or resultant incipient injury. The neuromonitoring clinician is bound by a technical role of set-up and signal attainment/optimization due to the in-person capacity, with limited interpretation.

Known supply kits have numerous problem(s) in the clinical healthcare setting/field, which are addressed by the present invention, as discussed in detail below. For example, some of the problems include excess of sterile supplies and material waste; communication errors with surgeon(s) by standardizing surgeon/case-type modality preferences; lack of ergonomic environment in the workplace, lack of adequate and quality of materials (solves error, forgetfulness, inadequate supplies; inconsistencies of obtaining multiple products per surgical procedure, for example, looking for tape and bite blocks (and other material resources included) in a hospital; inconsistencies among facility-supplied resources; bodily injury of needle sticks upon removal/dislodgement to self/others; fragmented process of clinical care among technicians; the challenge to order materials by the clinician (e.g., the time it takes to order each material category separately in anticipation of every possible procedure that could be monitored); and practical problems of technical jobs in the surgical/healthcare environment.

Therefore, there is a need for a supply kit that standardizes storage and access to the components for competent care in intraoperative neuromonitoring and that addresses, at a minimum, the above problems and shortcomings associated with prior art supply kits.

As discussed below, there have been attempts in the prior art to address the foregoing problems of providing a supply kits that can not only store supplies for a medical care, such as neuromonitoring but also provide functionality for safely receiving and storing used needles as well as providing a laptop work surface.

As to various laptop stands available on the market, most prior art stands are metal or plastic and are simply made for that sole purpose. However, these are generally not ideal for a neuromonitoring environment as they are bulky. There are some that are foldable and durable, however our goal is to make something compact and single use/worry free. The idea is that the clinician should not worry about taking anything extra after the case is done which is exactly our aim. The prior art is devoid a laptop stand that be easily and quickly assembled, then used and then easily throw it out. Finally, these alternatives are often not cheap and companies will not typically be willing to purchase these for clinicians and furthermore replace them should that be required.

Convertible boxes are also known in the art. For example convertible pizza boxes are known to house/contain pizza but can also be converted for the additional purpose of a pizza stand. Such a pizza box is foldable and can be put together to make a laptop stand, however, these stands are complicated to set up and expensive to manufacture.

For example, patent publication TWD2141685 utilizes a cardboard as a stand, reusing the cardboard that a certain type of notebook laptop comes with (packaging). However, its purpose is “environmental protection cooling” that also serves as a stand.

Prior art documents US20090321605A1, D84286251 and USD714302S1 provide a stand but do not include container storage functionality.

As to neuromonitoring kits, there also have been attempts in the prior art. For example, there is the SI-BONE iFuse Implant System neuromonitoring kit that has particular use for Minimally Invasive Sacroiliac Joint Surgery. Also, the NvM5 Machine includes “modules” for each modality such as SSEP module box & MEP/EMG module box. However, the kit is only by modality with limited functionality rather than an all-encompassing kit. Also, this prior art kit requires specific instrumentation/software, and the like so it cannot be easily utilized by all hospital/surgeons/clinicians/neuromonitoring companies. Also, it is known that surgeons rarely use these systems and clinicians have to be heavily trained. Therefore, it is preferred that the kit that is more user friendly and has universal capability.

Also, neuromonitoring system are well-known, such as seen in US Published Patent Document US20160278649A1, which teaches a machine that is directly related to the NvM5 (discussed above) that includes surgical instrumentation, rather than the providing of actual needles, bite blocks, and other neuromonitoring resources.

Still further needle collection boxes are also well known. For example, Chinese patent document CN109621098A teaches a syringe separating and reclaiming device that automatically separated recycling device of a biohazard waste recycling mechanical technique; fits the separating mechanism of the needle as a depository.

These solutions are not enough.

In view of the foregoing, there is a demand for a supply kit for medical care, preferably for neuromonitoring, that can store supplies, provide a housing to hold supplies for the neuromonitoring care, provide a container for receiving and storing used needles until they can be properly and safely disposed, all while providing a kit that can be converted to provide a work surface, such as for a laptop.

### SUMMARY OF THE INVENTION

The present invention preserves the advantages of prior art storage containers and supply kits. In addition, it provides new advantages not found in currently available container and kits and overcomes many disadvantages of such currently available—containers and kits.

The invention is generally directed to the novel and unique medical supply kit that, which can be used for any medical care, is specifically suited for neuromonitoring medical care.

More specifically, the neuromonitoring supply kit of the present invention is, for example, to be used directly by persons providing neuromonitoring services, both within an intraoperative and neurodiagnostic environment.

First, the supply kit of the present invention provides a container that contains medical care items. This may varying depending on the medical care to be delivered.

By way of background, such a supply kit can be used to facilitate intraoperative neuromonitoring is aimed at protecting the patient's nervous system during surgical procedures that place it at risk. Such surgical procedures include but are not limited to spine and brain/skull-base, peripheral nerve, vascular, and otolaryngology surgery. In this setting, various standard tests, can be included, such as Somatosensory Evoked Potential; Transcranial Motor Evoked Potential; Train of Four; Triggered and Spontaneous Electromyography; Electroencephalography. The following advanced tests/modalities conducted intraoperatively may also be provided, such as Brainstem Auditory Evoked Potential; Visual Evoked Potential; Electrocochleography; Phase Reversal; and Cortical Motor and Sensory Mapping. Also, various neurodiagnostic monitoring services may be included, but are not limited to Nerve Conduction Studies; Transcranial Doppler; Epilepsy Monitoring; and Ambulatory and Long-Term Electroencephalography

The neuromonitoring supply kit of the present invention may be used within any healthcare setting that utilizes intraoperative neuromonitoring and neurodiagnostic services. Most commonly, these kits will be directly in use by neuromonitoring clinicians who provide their services (in-house or contractual) in teaching and non-teaching hospitals of any size, encompassing trauma centers as well as non-for-profit and for profit organizations.

Outside of use within settings where direct health care is provided, the supply kit of the present invention, for example, can be used by academic institutions such as those offering a degree in surgical neurophysiology. Moreover, the supply kit of the present invention can be used as part of employee onboarding/training programs and continued education pathways. The supply kit of the present invention can be used within these settings both nationwide and globally. The present invention can also be used outside of a clinical setting and within an academic setting (e.g., an institution offering a degree in surgical neurophysiology). Thus, it can be used to teach academic students and future clinicians.

In general, the present invention may be configured in many different ways and still be within the scope of the present invention. For example, the neuromonitoring kit of the present invention first provides storage for the above-mentioned tests in a compact and easy to carry form. Also, the kit of the present invention may be reconfigured into a stand, which is particularly configured for supporting a computing device, such as laptop computer, table computer, and the like. Still further, the kit of the present invention can also be reconfigured into a device for the collection of used needles. For such needle collection, the present invention may be configured in at least two embodiments for this purpose.

It is, therefore, an object of the present invention to provide bi-functional utility, and inherent tangible/intangible benefits due to the invention's manufacture and presence.

It is a further object of the present invention to provide a neuromonitoring supply kit that can store commonly needed supplies and test equipment in a container device; convert that container device into a computer device stand; as well as convert the container device into a needle collection vessel for safe disposal of used needles.

The following are additional objectives of the present invention. These include reducing labor costs for clinical and surgical set up; surgeon satisfaction with customizable kits; clinician integration into care; assurance and quality of materials included; kits allow clinicians to be “always there” when monitoring is needed; ideal for add-on, or trauma procedures (ready-to-go); needle-count confirmation and minimization of needle-injury risks; customization for clinician by case/by surgeon/by hospital; shipped directly to clinician; standardizing the resources for competent intraoperative neuromonitoring; educational awareness in each kit; convenience of having everything in one place/ready to go; and empowers clinicians to have an active role, as opposed to automated systems by competitors. The present invention is also well-suited to hold and transport sterile supplies and to be safely disposed of in accordance with OSHA compliant receptacle due to its deconfiguration.

It should be noted that while the present invention has particularly applicability is provided a supply kit for neuromonitoring medical care, the present invention may configured to provide multifunctional medical supply kit for any type of medical care and still be within the scope of the present invention.

### BRIEF DESCRIPTION OF THE DRAWING FIGURES

The novel features which are characteristic of the present invention are set forth in the appended claims. However, the invention's preferred embodiments, together with further objects and attendant advantages, will be best understood by reference to the following detailed description taken in connection with the accompanying drawings in which:

FIG. 1A shows a front perspective view of the neuromonitoring supply kit of a first embodiment of the present invention in a closed condition;

FIG. 1B shows a rear embodiment of the first embodiment of FIG. 1A;

FIG. 1C shows a dieline of the supply kit of the first embodiment of the present invention when laid flat;

FIG. 1D shows a top view of the embodiment of FIG. 1A;

FIG. 1E shows a bottom view of the embodiment of FIG. 1A;

FIG. 1F shows a side view of the embodiment of FIG. 1A;

FIG. 1G shows a front view of the embodiment of FIG. 1A;

FIG. 1H shows a rear view of the embodiment of FIG. 1A;

FIG. 1I shows a right side elevational view of the embodiment of the present invention of FIG. 1A in a partially open condition with the front flap in the process of being opened;

FIG. 1J shows a left side elevational view of the embodiment of the present invention of FIG. 1A in a partially open condition with the front flap in the process of being opened;

FIG. 1K shows a front elevational view of the embodiment of the present invention of FIG. 1A in a partially open condition with the front flap in the process of being opened;

FIG. 1L shows a front elevational view of the embodiment of the present invention of FIG. 1A in an open condition with the front flap further opened to permit access to the interior of the container;

FIG. 1M a side elevational view of the embodiment of the present invention of FIG. 1A in an open condition with the front flap further opened to permit access to the interior of the container;

FIG. 2 shows a front elevational view of the embodiment of the present invention of FIG. 1A in an open condition with the front flap further opened to permit access to the interior of the container where the container is filled with various medical-related supplies and tests, such as those for neuromonitoring;

FIGS. 3A-G show the conversion of the container of the kit of the present invention into a stand for a computer device;

FIGS. 4A-C show the details of the conversion and use of the container of the first embodiment of the present invention of FIG. 1A for needle collection;

FIG. 5A shows a front perspective view of a second embodiment of the neuromonitoring supply kit of the present invention;

FIG. 5B a left front perspective view of a second embodiment of the present invention of FIG. 5A in an open condition with the front flap further opened to permit access to the interior of the container (contents of supplies and tests not shown for ease of illustration);

FIG. 5C a right side perspective view of a second embodiment of the present invention of FIG. 5A in an open condition with the front flap further opened to permit access to the interior of the container (contents of supplies and tests not shown for ease of illustration);

FIG. 5D shows a top view of the dieline of the supply kit of the second embodiment of the present invention when laid flat;

FIG. 5E shows a bottom view of the dieline of the supply kit of the second embodiment of the present invention when laid flat;

FIG. 6A shows the configuration of the second embodiment of the present invention into a needle collection vessel where the front flap is opened to reveal the opening on the front of the container to receive needles therethrough;

FIG. 6B shows the front flap in the process of being retained in an open condition of the second embodiment;

FIG. 6C shows a side perspective view of the container in an open condition to fully convert the kit to a needle collection device in accordance with a second embodiment of the present invention;

FIG. 6D show a top perspective view of the container in an open condition to fully convert the kit to a needle collection device in accordance with a second embodiment of the present invention; and

FIG. 6E side perspective view of the container of a second embodiment of the present invention configured with a cord to assist with carrying the kit of the second embodiment of the present invention.

### DESCRIPTION OF THE INVENTION

The supply kit 10 of the present invention provides, as a main function, a container for housing various medical supplies 12, such as for carrying out neuromonitoring. While the kit 10 of the present invention is particularly well-suited for containing and carrying various supplies 12 and testing items, it may be loaded with items 12 for any medical procedure or purpose and still be within the scope of the present invention. By way of example, as seen in FIG. 2, the following items 12 may be included in the kit 10 of the present invention as a necessary kit item, such as for neuromonitoring medical care: the box of the present invention with design (Small)—4.5″×7″×2.5″ Mailer Boxes; the Box with Design (Large)—9.5″×7″×2.5″ Mailer Boxes; Clinician Guide—Muscle Innervation/Kit Instruction Guide; Instruction Labels (1-2)—1-2 Adhesive Sticker Labels; Left (L)—Laterality Label—Adhesive L sticker; Right (R)—Laterality Label—Adhesive R sticker; Mini Scissors—3½″ Mini Scissors (Orange); Needle Bags—Plymor Heavy Duty Plastic Reclosable Zipper Bags, 4 Mil, 6″×4″; Neuroplex Disposable Pen—Washington Pen with Black Ink Digital Full, Color Wrap with Logo; Box Stickers, Circles—Double Sided Foam Tape Strong Pad—Mounting Adhesive (3M); Alcohol Pads—Alcohol Prep Pad—2 Ply, Medium; Soft-Cloth Surgical Tape—3M Medipore H 2″×10 Yard Hypoallergenic Soft Cloth Surgical Tape, Special Pack of 12 Roll; Clinician Number Cards—Customized Contact Info Cards for Clinicians

Also, various surgical supplies 12 are preferably included in the kit 10, such as a subdermal Needle, Twisted Pairs—14 mm×28 ga 2.5 m Lead Twisted Pairs (6-colors); surface Electrode, Sticky (Sm)—15 mm×20 mm (2.5 lead) Tab, Electrode-Paired Stimulating Sticky Pad; Surface Electrode, Sticky (Lg)—a 20 mm×25 mm (2.5 lead) Tab, Electrode-Paired Stimulating Sticky Pad; Head-Lead (6)—RLI Multi-Lead Subdermal Twisted 6—Lead Head Lead; and Bite Block—Disposable Protection for Teeth.

A paper clinician guide with basic brain and spine anatomy as well as step-by-step description of how to utilize the box for its other dual functions may also be included in the kit 10. The box/kit 10 is preferably delivered to the clinician in tamper proof packing and a label indicating the surgery type for which the kit may be used. Each clinician will bring this box into the operating room, open the kit 10, remove its contents 12, and use it as needed throughout the case.

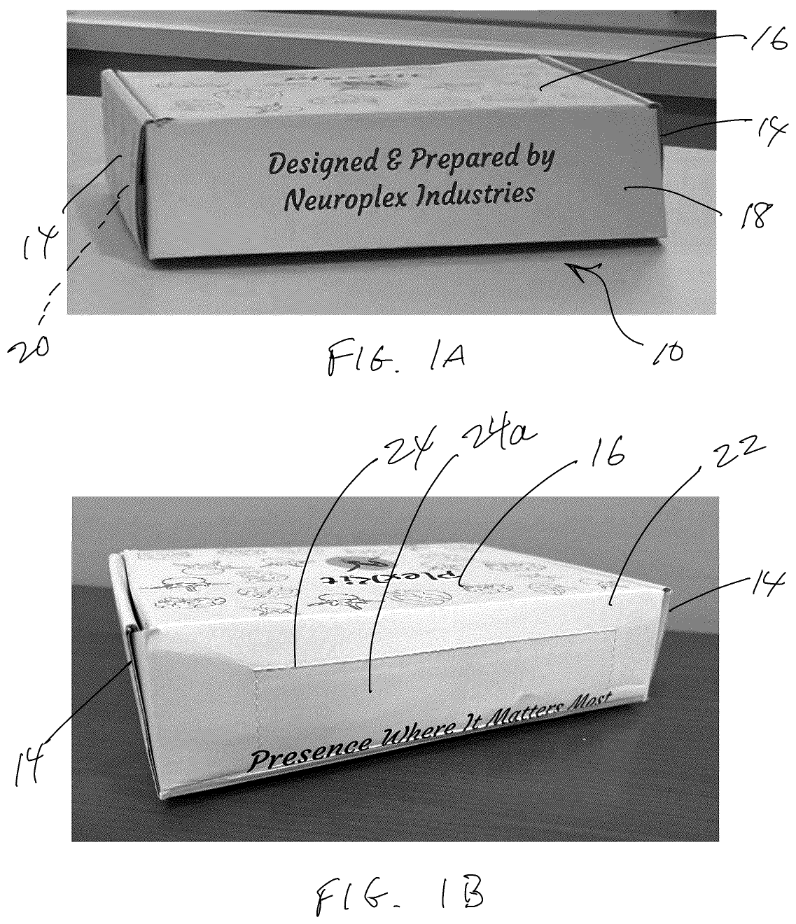

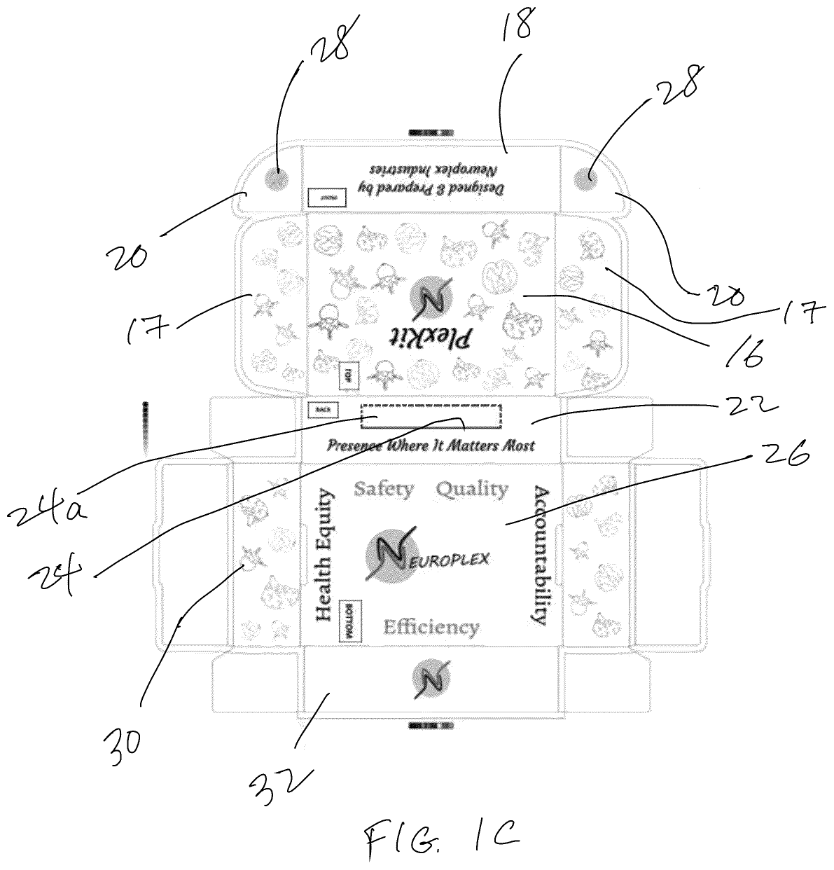

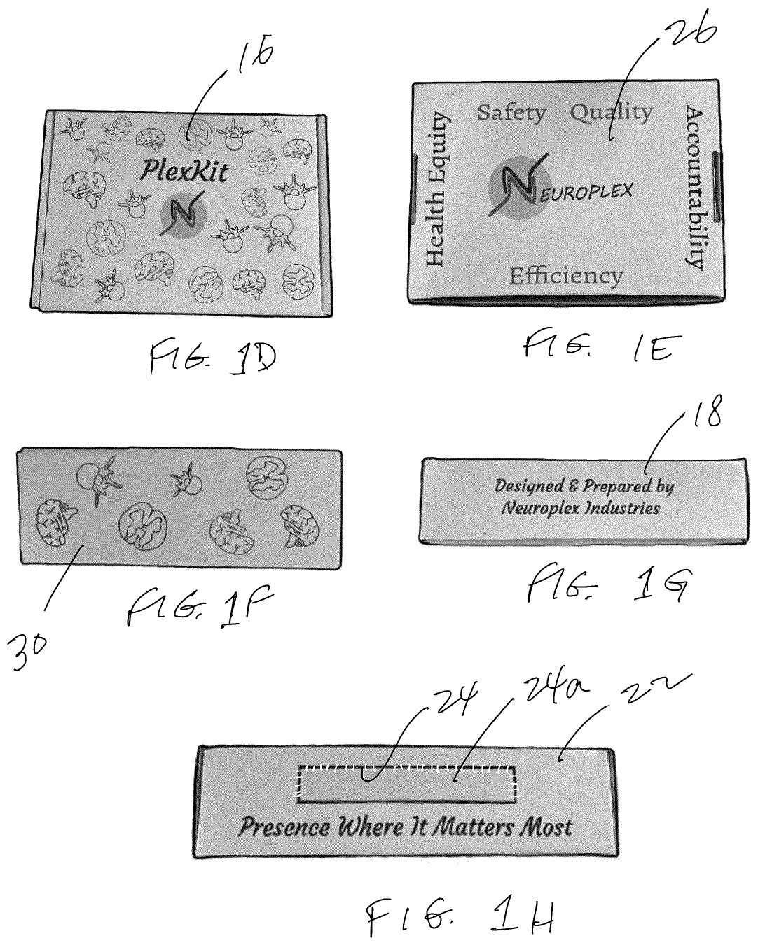

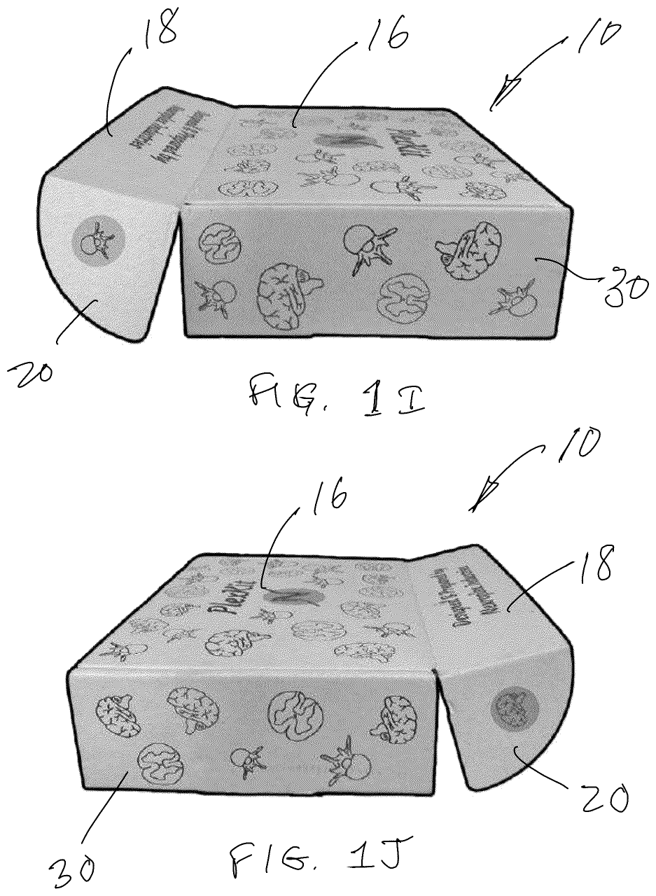

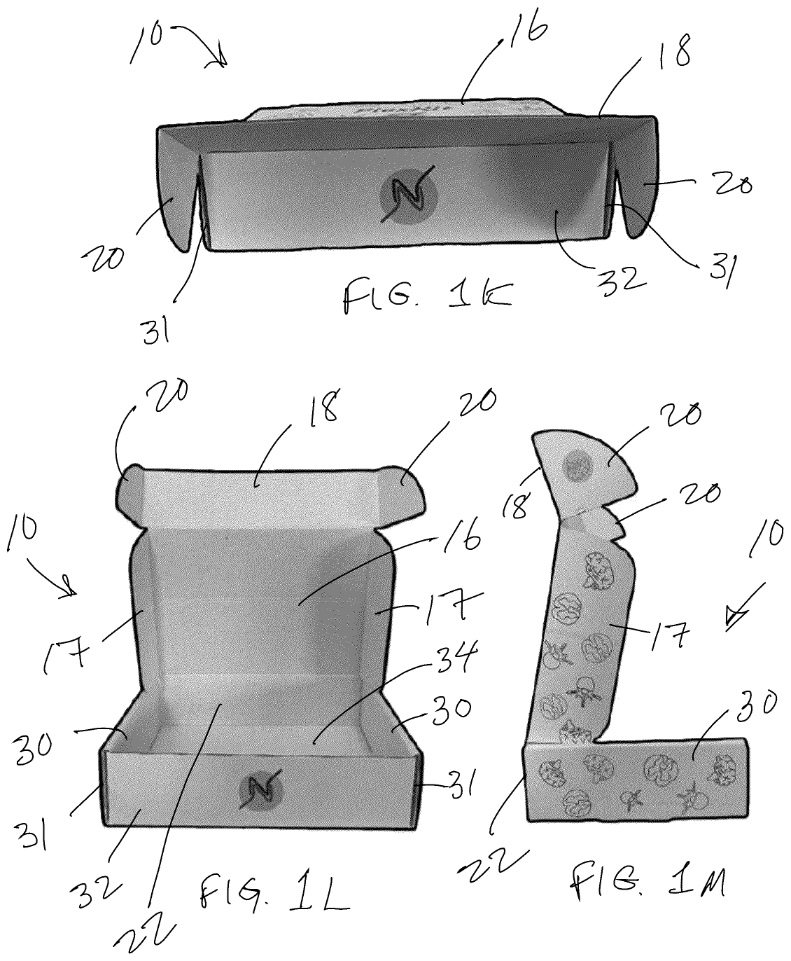

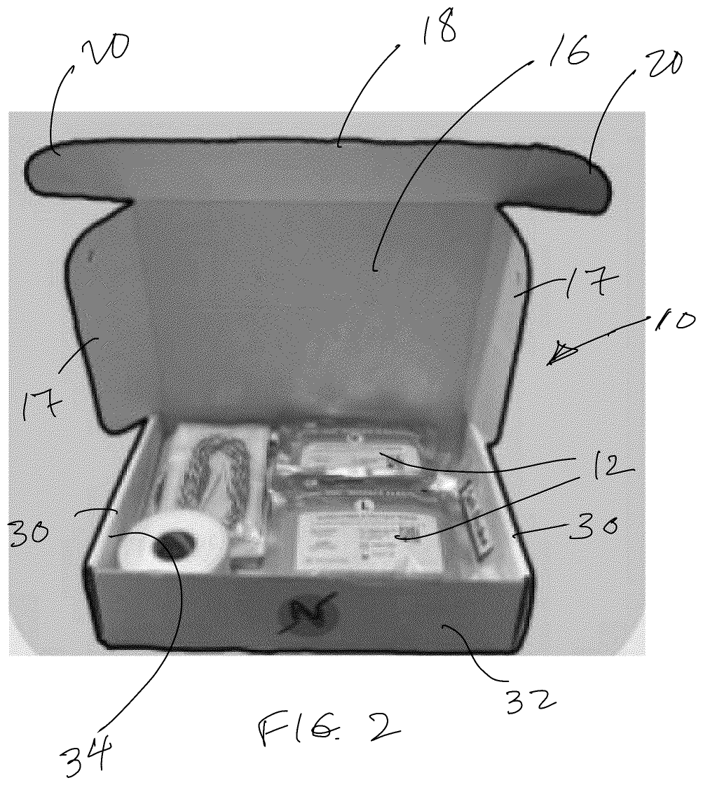

Turning next to the structure and configuration of the supply kit, FIGS. 1A-M show the supply kit container in different conditions as well as being laid flat before it is assembled into a container box.

Turning first to FIG. 1A, a front perspective view of the neuromonitoring supply kit of a first embodiment of the present invention in a closed condition is shown. FIG. 1B shows a rear view of the first embodiment 10 of FIG. 1A. The container kit 10 of a first embodiment of the present invention includes side walls, a top panel 16 connected to a front flap 18 with side tabs 20 where the side tabs 20 can be seen in FIGS. 1I and 1J, for example. In FIG. 1B, the rear panel 22 is shown to include a needle entry opening 24 is provided as a partially perforated opening where, for example, three sides are perforated so the opening 24 can be provided while leaving a needle opening door 24a, which could be closed at a later time.

FIG. 1C shows a dieline of the supply kit 10 of the first embodiment of the present invention when laid flat in different variations, before it is assembled, namely, the dieline of the kit/box 10 to show kit's outside design. Front 18, top 16, bottom 26, left, and right panels of the kit 10 after it is put together. The dimensions of this configuration of the container box 10 may vary but are preferably: 9.50″×7″×2.5″ While the same design will be utilized for all kits 10, dimensions thereof can be adjusted to meet the needs of the clinician (i.e., to include more/less accessories) and the needs of the particular medical care being delivered with the assistance of the kit 10 of the present invention. Various optional indicia may be provided on the box as a company branding and company values. Seen on the top panel 16 are the kit name (PlexKit), minimized company branding, and anatomical structures for cosmetic purposes. Seen on the back panel 26 of the box 10 are further indicia, such as more company branding and a rectangular shape for use of the kit's additional dual function (described in detail below). Other similar indicia may be provided on other panels of the box.

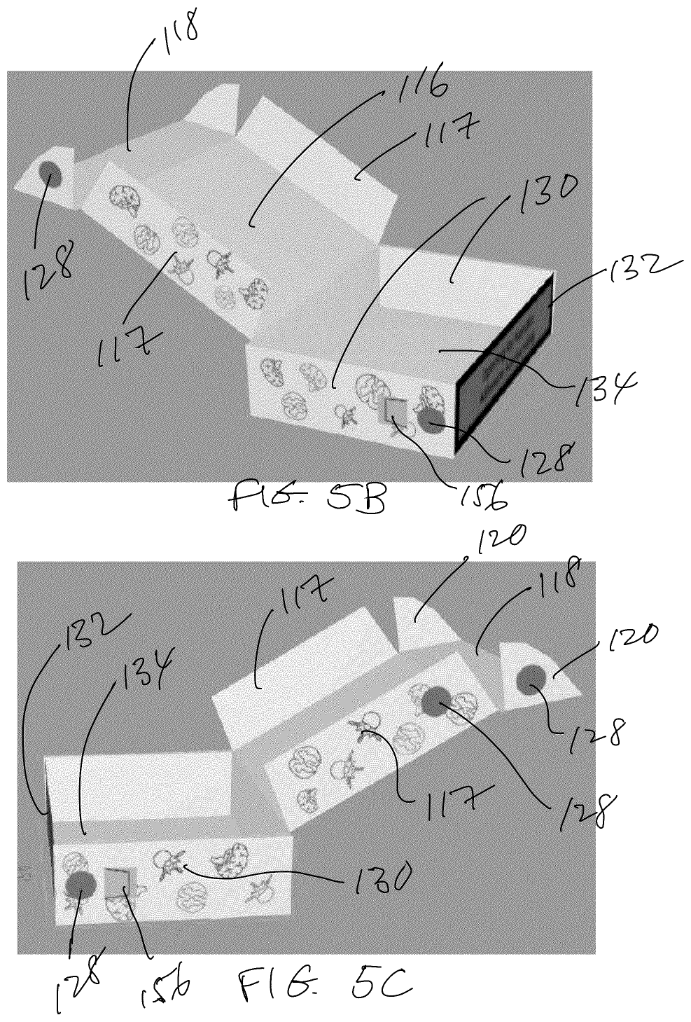

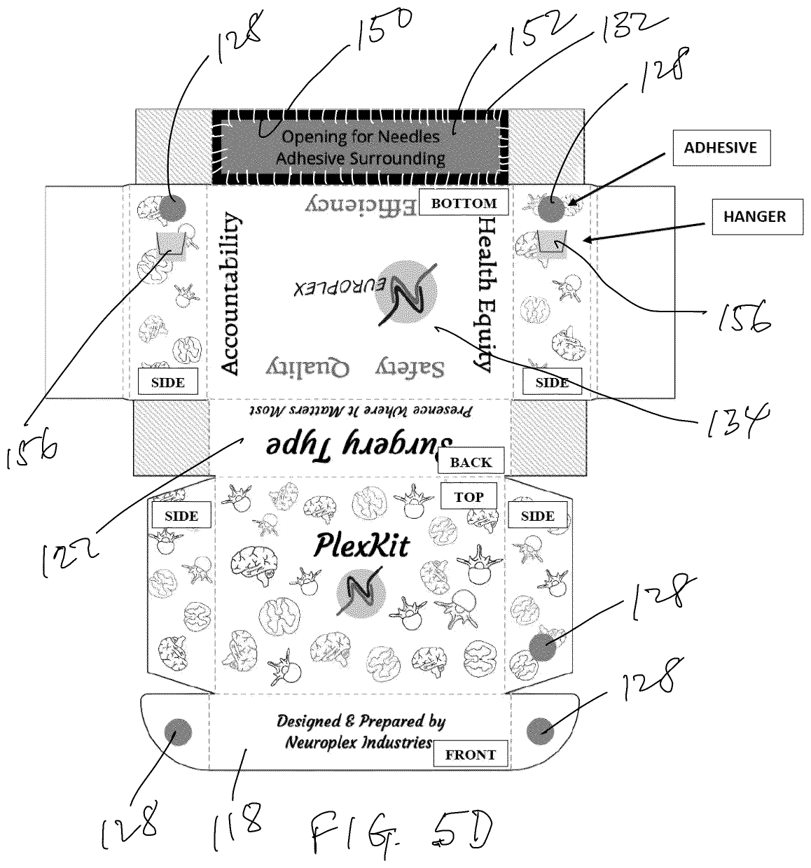

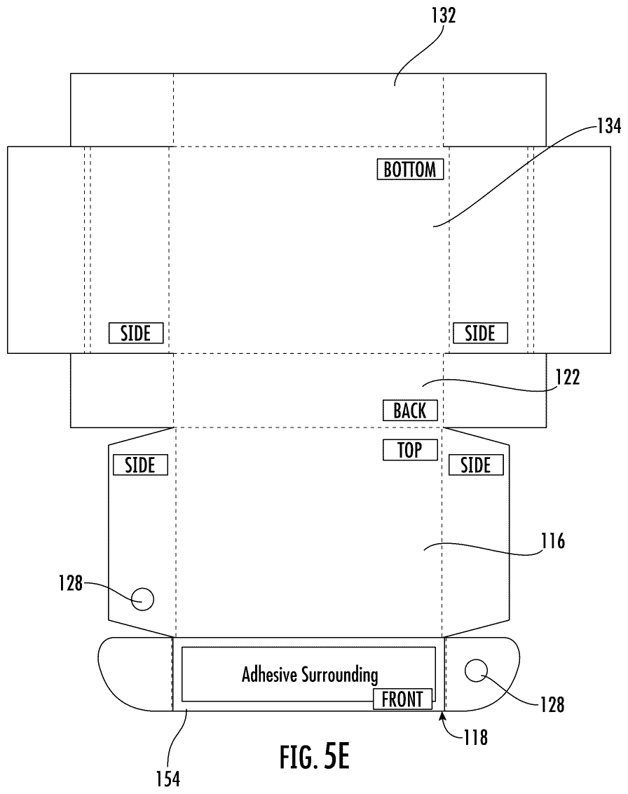

It should be noted that the dieline sheet of FIG. 1C (as well as those shown in FIGS. 5D, 5E) are just one way to form the end shape of the kit 10 of the present invention. It is envisioned that there are many different ways to creates and form the end kit by different folding of various panels. As indicated below, the kit may not even employ a folding design, instead the final kit maybe formed directly into a final kit shape.

The laid flat view of FIG. 1C shows all of the panels and, namely, the needle door 24a that will be present on a rear panel 22 of the container kit 10 after the container kit 10 is assembled. Such a rear view with needle collection door/port 24a can be seen in FIG. 1H. Fasteners 28, such as hook and loop material or adhesive tape, are provided on tabs 20. Additional panels 30, 32 are provided to enable construction and folding of the kit 10 into a final form. Side flaps 17 are connected to top panel 16.

Still further, FIG. 1D shows a top view with top panel 16 of the first embodiment of FIG. 1A where FIG. 1E shows a bottom view with bottom panel 26 of the embodiment of FIG. 1A. FIG. 1F shows a side view with side panel 30 of the embodiment of FIG. 1A while FIG. 1G shows a front view with front flap 28 of the embodiment of FIG. 1A. As noted above, FIG. 1H shows a rear view with rear panel 22 and needle port 24 of the embodiment of FIG. 1A with the partially perforated needle door/port 24a for use when the first embodiment 10 of the present invention is configured as a needle collection vessel.

FIG. 1I shows a right side elevational view, FIG. 1J shows a left side elevational view, and FIG. 1K shows a front view of the first embodiment 10 of the present invention in a partially open condition with the front flap 18 in the process of being opened (or closed) where the tabs 20 of the front flap 18 are disengaged with the rest of the container box 10, namely side panels 30. As can be seen in FIG. 1K, the front panel 32 and the side panels 30 define left and right gaps 31 on opposing sides of the container box 10 to receive the tabs 20 of the front flap 18 in a typical fashion as a typical cardboard box. Therefore, the tabs 20 on the front flap 18 can be inserted or removed from the respective gaps 31 in the box 10 to secure the front flap 18 in a closed condition, as in FIG. 1A.

Turning now to FIGS. 1L and 1M, a front elevational and side elevational views are respectively shown of the embodiment 10 of the present invention of FIG. 1A in an open condition with the top panel 16 and front flap 18 further opened to permit access to the interior 34 of the container. FIG. 2 shows various surgical supplies and/or medical tests 12, generally referred to as supplies, of the nature described above in detail. As noted above, these supplies and items 12 are shown as examples only of what can be loaded into the container box 10 of the present invention. With the container box 10 opened up, as in FIG. 2, the medical supplies 12 can be easily loaded into the box 10 and then the top panel 16, front flap 18 and tabs 20 secured in place to contain the supplies 12 therein. As can be understood, the kit 10 of the present invention is preferably shipped to the consuming medical care giver (e.g., clinician) already loaded with various medical supplies 12. It is also possible to ship the container box 10 empty to the care giver to permit that care giver to load the container box with their own desired supplies 12. The present invention can be converted/reconfigured as a computer device stand as well as a needle collection vessel, as will be described in detail below.

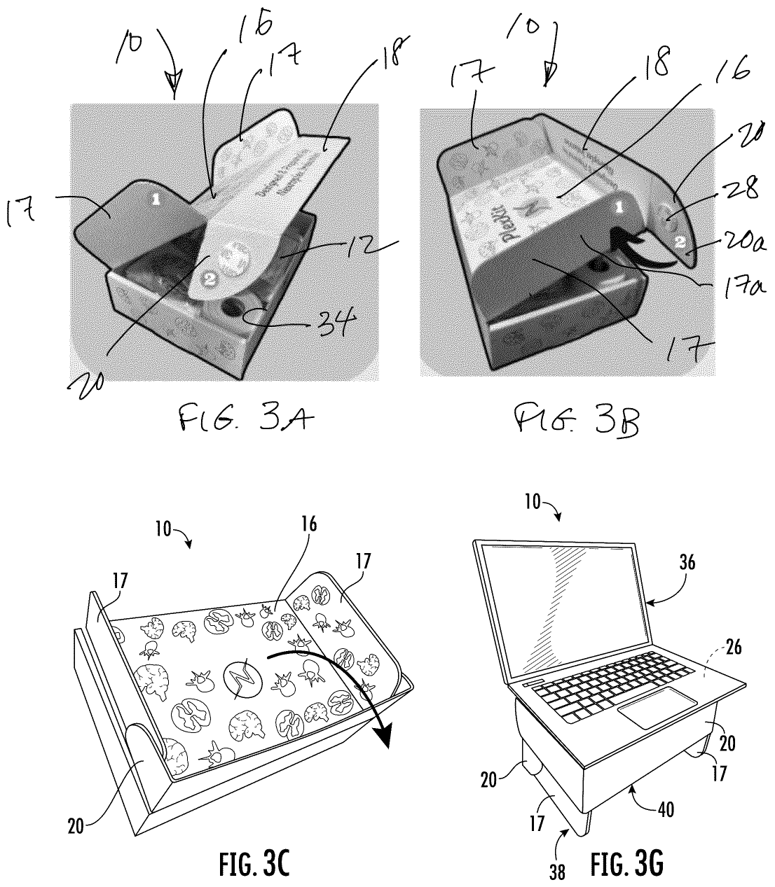

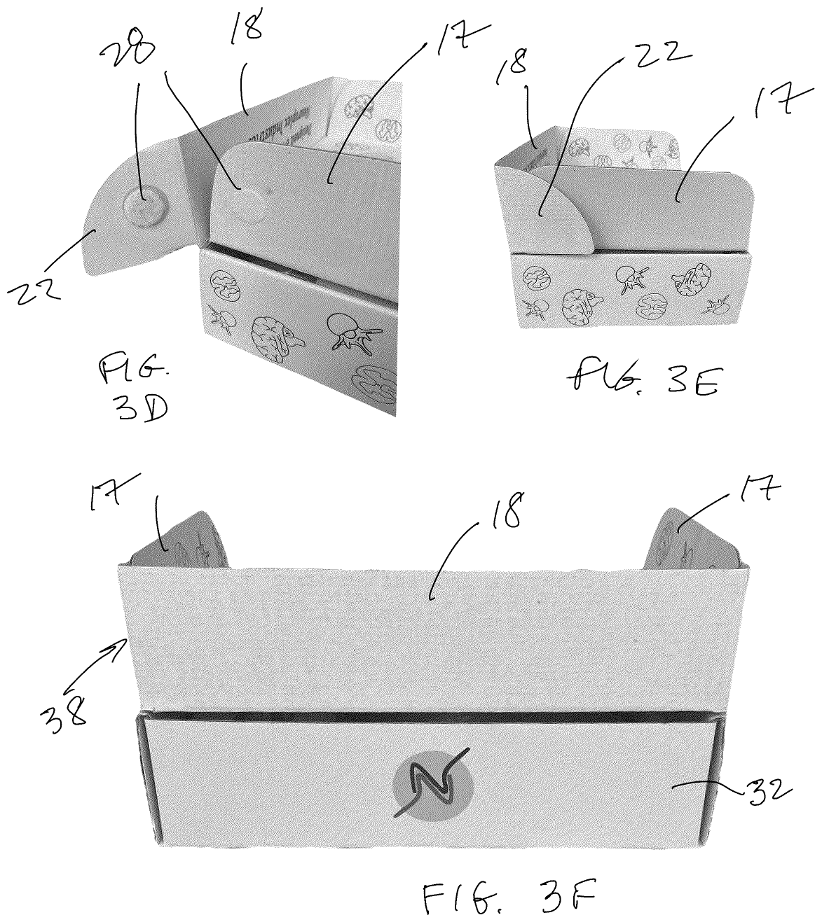

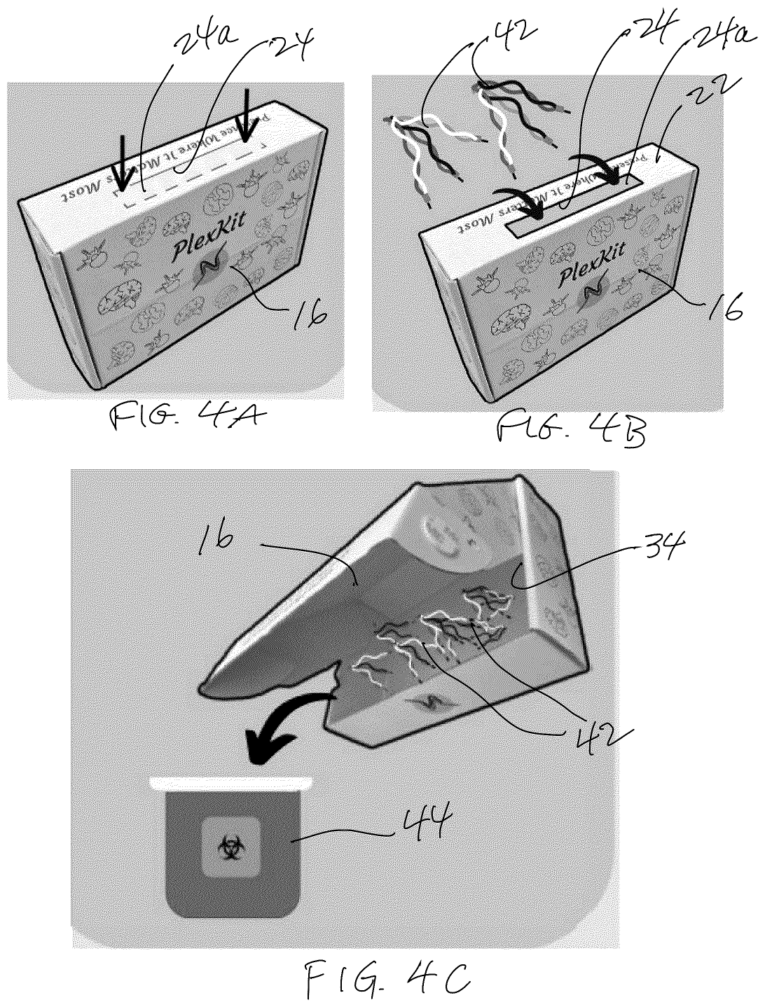

As stated above, the kit 10 of the present invention can be converted into a computer device stand or a needle collection vessel. First, FIGS. 3A-G show the steps of conversion of the container box 10 of the first embodiment of the present invention into a computer device stand. For example, computer devices, such as a laptops, tablets, and the like, generally referred to as 36, may benefit from the computer device stand configuration of the first embodiment 10 of the present invention.

When the container box 10 is converted/reconfigured into the laptop stand, it offers, for example, five inches of elevation, providing a more ergonomic working environment for the clinician or medical care giver when using device 36. The dimensions and height of the stand portion 38 may be modified to suit the given application and/or size of the supply kit housing box 10.

As in FIGS. 3A-G, the method for converting the supply kit 10 of the present invention to provide a computer device stand 38 includes first orienting an unopened kit box 10 in center-front position with a side flap 17 oriented toward the user. The cover top panel 16 is opened and the front flap 18 and side tabs 20 and the side flaps 17 are disengaged with the bottom tray-like portion 30, 32, 22 of the container box 10. As in FIG. 3A, the side flaps 17 are hinged upwardly. In FIG. 3B, the side flaps 17 and the front flap 18 are hinged upwardly so they are approximately perpendicular to the top cover panel 16. Still referring to FIG. 3B, the side tabs 20 on the front flap 18 are hinged rearwardly so an inner surface 20a of the tabs 20 on the front flap 18 communicate with the forward portions of the inner surfaces 17a of the side flaps 17. The tabs 20 on the front flap 18 and the side flaps 17 are preferably releasably secured to each other by some type of fastener 28, such as hook and loop material on the two mating surfaces or adhesive material on one or both of the two mating surfaces of the tabs 20 and side panel 17.

As seen in FIG. 3C, a three-sided riser stand portion 38 is now provided by the reconfigured side flaps 17, front flap 18, top panel 16 and tabs 20 on the front flap 18 at a location on the top of the container box 10.

FIG. 3D shows a close-up view of the releasable engagement of the tabs 20 on the front flap 18 with respective side flaps 17 with fasteners 28 while FIG. 3E shows a close-up view of the front flap 18 and side flaps 17 secured to each other with the tabs 20 of the front flap 18 to provide a three-sided riser portion 38. FIG. 3F shows a further view of the riser/stand portion 38 that has now been configured to enable the container kit 10 to serve as a computer device stand.

Thus, the entire, now reconfigured box 10 with computer device stand 38, is now flipped upside down, as in FIG. 3G, to cause the three-sided riser portion 38 to now be on the bottom of the device 10 whereby the bottom surface 26 now provides a support surface for the computer device 36, while also providing a storage space 40 therebelow for other items while the medical care is being delivered.

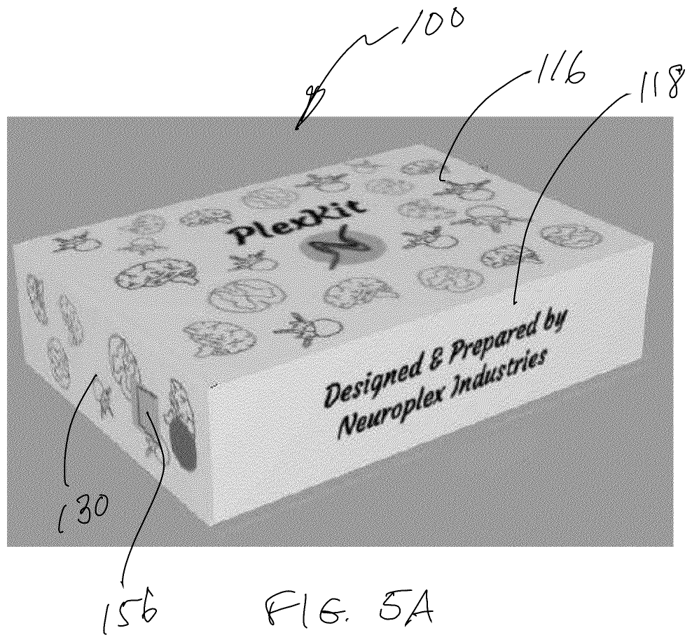

Referring now to FIGS. 4A-C, details of the ability of the supply kit 10 to be converted or transformed into a needle collecting container is shown. The supply kit 10 is first emptied to remove the contents 12 thereof. Such contents 12 are being used anyway in connection with rendering the medical care. As in FIG. 4A, the care giver, such as a clinician, pushes the door material 24a in at the perforated rectangle at the back panel 22 of the box 10, a space opens up allowing the clinician to insert needles 42 collected from the patient as the end of the procedure. It should be understood that the perforation preferably only present at three of the four lines of the rectangular opening. As a result, a material flap 24a or door is left remaining attached to the body at panel 22 of the kit and serves as a secondary protective measure against needles falling out of the box 10. This perforated flap or door 24a increases the safety for secured sharps disposal whereby the kit 10 is customizable and intended for one-time use only.

If the container box 10 is configured as a computer device stand with riser portion 38, the box 10 is reoriented 180-degrees toward the user, or reoriented stationary box in laptop-stand configuration in clockwise manner, if viewed from the side. The tabs 20 on the front flap 18 are unfastened from the side flaps 17 on both sides.

The front flap 18 is pulled downward so that the tabs 20 on the front flap 18 on the right and left side, respectively, are no longer touching the side flaps 17. The top cover panel 16 is directed upright into erect position. The side flaps 17 are bent inwardly from 180-degrees (flat, free-form) to a final 90-degree angle, congruent with the perimeter of the base (bottom). The tabs 20 on the front flap 18 are respectively folded inwardly and downward and are then re-affixed internally on their mating portions on the side flap 17. The tabs 20 may be secured to an inner or outer portion of the side flaps 17 or one tab may be secured on an inner side while the other tab 20 is affixed to an outer side of the respective side flaps. The main top panel 16 is moved back down toward the base (bottom) to re-close the container 10 into a condition see in FIG. 1A. With medium, forceful pressure, the newly configured top is pushed into the base (bottom).

As in FIG. 4A, the container box 10 is reoriented with top panel 16 of the box 10 facing toward the user with the rear panel 22, with the partially perforated opening 24, facing upwardly. Thus, the user next gently presses into the door 24a1 at the partially perforated opening 24, namely, along the three-sided perforated edges located on rear panel 22. As a result, the door flap 24a hinges inwardly into the container box 10, as seen in FIG. 4B thereby providing a passageway for the receipt of used needles therethrough. Since the container box 10 is closed, as in FIGS. 4A, B, the needles 42 inserted through the needle opening 24 with flapped door 24a, the needles 42 inserted therein tend to desirably stay inside the container box 10. When desired, the container box 10 can be emptied of the collected used needles 42, as in FIG. 4C, wherein the top panel 16 is simply hinged upwardly to expose the interior 34 of the box 10 at which time the used needle contents 42 may be emptied into a sharps container 44 for proper later disposal.

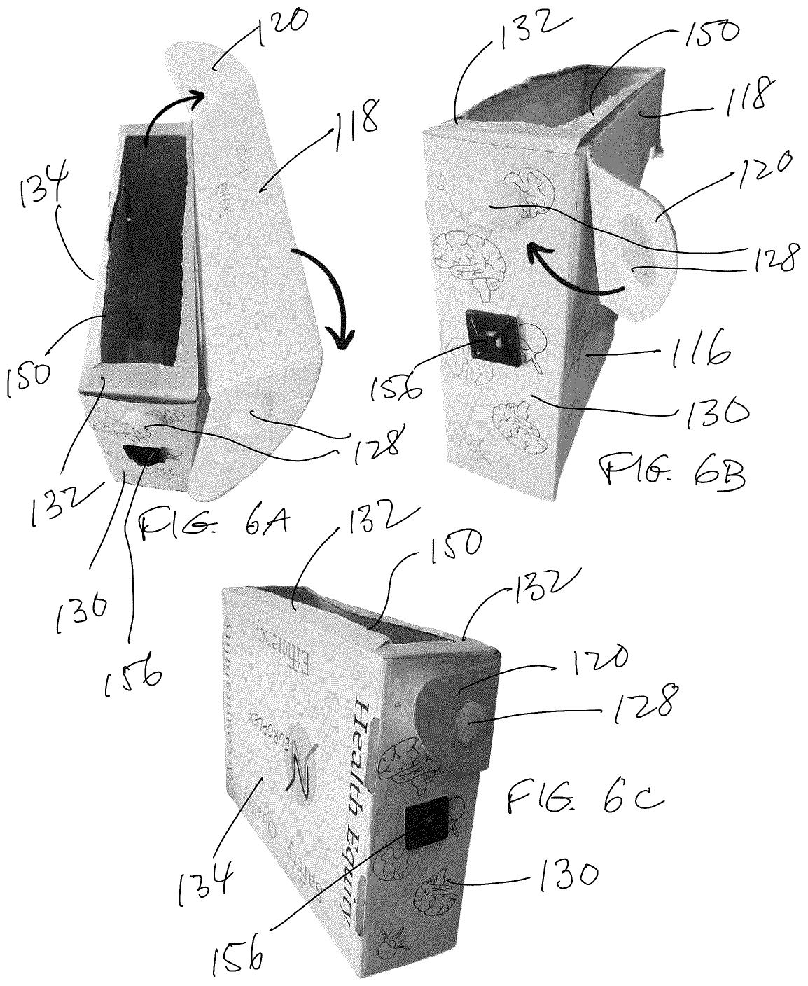

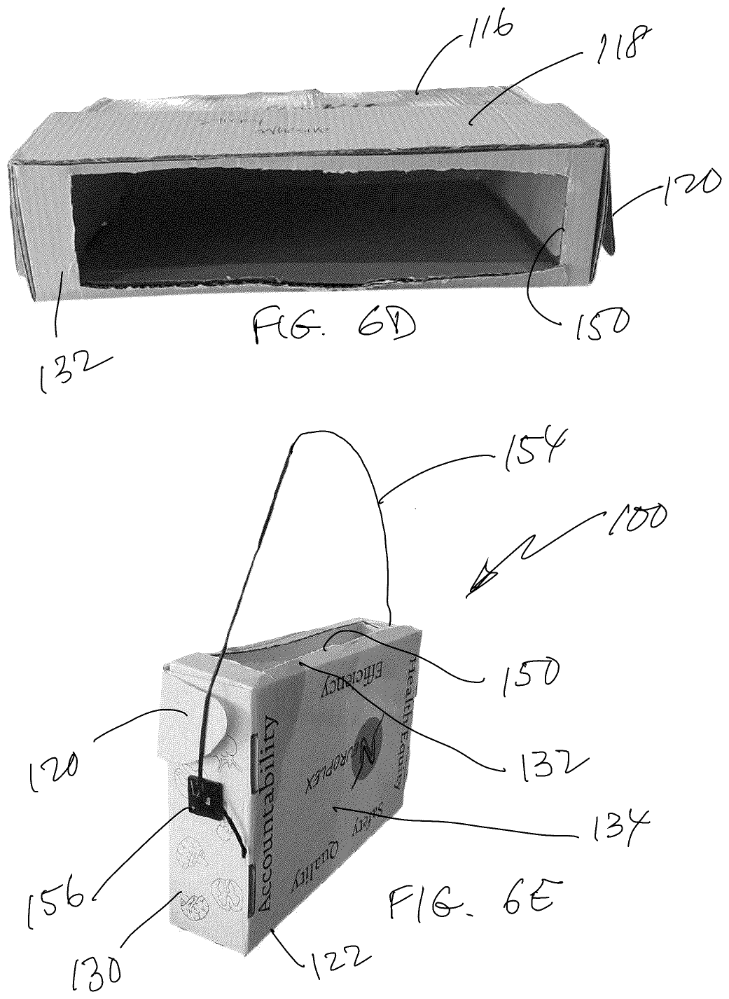

Turning now to FIGS. 5A-E and FIGS. 6A-E, a second embodiment 100 of the present invention is shown in detail. More specifically, the second embodiment 100 of the present invention converts into a computer device stand in the same fashion as the first embodiment 10 so there is no need to discuss this feature again in connection with the second embodiment 100. However, the second embodiment 100 provides an alternative configuration for converting the container box 100 into a needle collecting vessel.

Referring first to FIG. 5A, a front perspective view of the second embodiment of the neuromonitoring supply kit of the present invention while FIG. 5B shows a left front perspective view and FIG. 5C shows a right front perspective view of the second embodiment of the present invention of FIG. 5A in an open condition with the front flap 118 further opened to permit access to the interior 134 of the container 10 (contents of supplies and tests not shown for ease of illustration). FIG. 5D shows a top view of the dieline of the supply kit 100 of the second embodiment of the present invention when laid flat. FIG. 5E shows a bottom view of the dieline of the supply kit of the second embodiment 100 of the present invention when laid flat to further illustrate the location of the full pass through needle opening 150 in this embodiment 100 that is created after perforated panel 152 is knocked out and removed from front panel 132. It should be noted that the inside surface of the front flap 118 is provided with adhesive 154 such as an adhesive with peel-off release tape about the periphery of the front panel 132. As will be described below in connection with FIGS. 6A-E, this front flap adhesive 154 engages with the outer surface of the front panel 132, namely, the surface about the full needle opening 150, as seen in FIGS. 5B, 5D when needle collection is complete and there is a desire to seal up the container box 100 with used needles captured therein.

It should be particularly noted that this second embodiment 100 includes a full needle receiving opening 150 on the front panel 132 of the container box 100. The needle receiving opening 150 is perforated on all sides so the panel 152 can be knocked out completely to provide the desired full pass through opening 150.

The location and configuration of the needle opening 150 is different that the first embodiment 10 in that the needle opening 150 is completely opened without leaving a door or flap and the location of the needle opening is now through the front panel 132 instead of through the rear panel as in the first embodiment 10. FIG. 6A shows the configuration of the second embodiment 100 of the present invention into a needle collection vessel where the front flap 118 is opened to reveal the opening 150 on the front panel 132 of the container 100 to receive needles (not shown) therethrough. If not already knocked out, the needle collection panel 152 can be knocked out via the perforation to provide a large easy access opening 150 to receive used needles therethrough.

To facilitate and maintain access to the needle opening 150, the front flap 118 is preferably folded over to contact the top panel 116. The tabs 120 on the front flap 118 are reversed, as in FIGS. 6A, 6B and the folded toward a corresponding side panel 130 of the box 100, as in FIG. 6B, in particular. As in FIG. 6C, the tabs 120 are folded over so the mating fastening 128, such as hook and loop fasteners or other adhesive, is engaged so that the tabs 120 and front flap 118 are retained in place, as in FIG. 6C. Thus, in FIG. 6C, a side perspective view of the container of a second embodiment 100 is shown in an open condition to fully convert the kit 100 to a needle collection device. FIG. 6D shows a top perspective view of the container 100 in an open condition to fully convert the kit 100 to a needle collection device in accordance with a second embodiment 100 of the present invention while FIG. 6E shows a side perspective view of the container of a second embodiment 100.

Therefore, uniquely and as best seen in FIG. 6E, the second embodiment 100 of the present invention provides a temporary sharps-like container 100 that sits upright (sitting on the rear panel 122) to provide the needle receiving opening 150 facing directly upward for ease of insertion of used needles therein. FIG. 6E also shows the attachment of a hanger or carrying cord 154 which extends from one side of the container box to the other, such from the side panels 130. For example, a side clip or connector 156 is provided on the opposing sides 130 of the container 100 to receive free ends of a cord 154 or rope, which is preferably included inside the container 100 with the medical supplies when the kit 100 ships. This allows for the hanging or carrying of the kit 100 when configured as a collecting vessel, such as after all of the used needles and/or needle electrodes have been collected.

In view of the above, the supply kit 10, 100 of the present invention has numerous advantages that are attainable with the multi-functional utility and/or included resources. The present invention has numerous advantages and benefits, such as increasing the standardization of monitoring care where safety measures are standardized during neuromonitoring at various facilities and where the kit includes recommended muscles to monitor. Moreover, the present invention 10, 100 ensures that a surgeon gets a standardized monitoring set up for each common procedure. The present invention 10, 100 also reduces communication errors with surgeon(s) by standardizing surgeon/case-type modality preferences.

Also, supplemental labor costs are reduced by reducing technical set-up time, minimizes prior preparation of materials, and reducing supply-chain and logistical operations by implementing one-time use kits. The present invention 10, 100 reduces material waste and provides multi-function capability in a single devices that can provide a computer device stand and an electrode collector/transporter which results in the reduction of the lack of materials/resources at facilities/supply chain issues.

In view of the above, the present invention 10, 100 has many tangible benefits. For example, the present invention 10, 100 is compact, where a Surgical Neurophysiologist (SNP) can carry more kits at one time. It is convenient, where everything that is needed for the medical care, such a neuromonitoring. It is also more affordable than prior art devices.

The kit 10, 100 of the present invention also facilitates and supports training as it includes labels for needles and packaging that is straight forward by laterality and muscle which helps streamline the training process in a fast paced operating room environment.

As mentioned above, the present invention 10, 100 also streamlines supply chain processes for healthcare-to-vendor channels. For example, it provides back support where the container box can fold so that it doubles its height, such as to five inches. The present invention 10, 100 helps to keep the care giver organized by keeping everything in one place. The invention 10, 100 also folds to add back support functionality, which also creates a storage compartment keeping the working environment organized. The present invention helps clinician focus their attention on setting up a patient for optimal monitoring and spend more time on optimizing stimulation/recording parameters based on medical history rather that spending time on setting up.

Another advantage of the present invention 10, 100 is the standardization of health care to ensure that all patients get the same kind and level of care by standardizing the number/types of muscles being monitored based on the case. Further academic support is provided with muscle resources readily available where it mediates SNP & surgeon communication, precise analysis of neurophysiologic signals, as well as other tests, and correlation to levels at risk. Furthermore, needle safety is improved with the present invention where puncture-safe bags that connect to a wrist key chain that SNP has hold as they are removing needles from the patient. Quick and safe disposal minimizes the need to run back and forth to get rid of needles or from holding bunches of electrodes in one hand. It further minimizes risk of needles falling on the group or patient's bed as well as risk of needle stick in transportation. The transparent color of bags also adds the ability to count needles to ensure that needles placed in the PT were taken out. Bags used in accordance with the present invention can optionally have stickers on them that state “needle in; needle out” for further clarification of the handling of needles.

The present invention 10, 100 is easy to manufacture because of the nature of the materials used. There is ease of obtaining materials and manufacture thereof. The kit 10, 100 is easy to ship to clinician or other user. There can be repetitive and automated portions, with periodic adjustments of inventory/preferences.

The kit 10, 100 of the present invention is also very durable. Excellent durability for one-time use to maintain sterility and health precautions for the integrity of each surgical procedure. Malleable form and capacity of kit to allow dual-function, increasing the longevity/utility of the product. Additional layer (or multiple layers) of packaging/protection against anticipated needle puncture, shipping/transportation exposure, and other non-sterile environments, and the like. Also, the life of the kit of the present invention is envisioned as a one-time use, repeated/consecutive orders necessitating a continuous need/demand.

The present invention also enables time to plan where the duration of time it takes to derive a clinical protocol of grouped muscles, pulling the correct number of electrodes and necessary materials, confirmation of nervous system modalities tested, and subsequent communication to clinical staff and/or surgeons. This is an advantage that can be realized when the supply kit of the present invention is used.

The present invention 10, 100 also helps with time to unpack supplies. The duration of time it takes to unpack separate boxes and/or clinical resources from sterile packaging, obtain all necessary materials for the specific case, and re-confirm all materials are opened and gathered can be reduced when the supply kit of the present invention is employed.

Also, the present invention 10, 100 assists in time to organize for the clinician where the duration of time it takes to separate unpacked resources, organize into correct laterality and/or order (for clinical setup), and re-confirmation of resources needed for the surgical procedure is reduced when the present supply kit is used.

The supply kit 10, 100 of the present invention is preferably made of cardboard but may be made of other materials, or a combination of materials, depending on application and intended use at hand. For example, the kit 10, 100 of the present invention may be made of high density polyethylene plastic or other plastic materials. In sum, the present invention may be made out of any material that can be formed into the configurations of the present invention where the needle access openings can be formed and perforated or otherwise prepared for easy opening by the user. This material may be formed in any way, such as die cutting or laser cutting from a sheet of material. Also, the container box may be formed directly by molding or 3D printing to obviate the need for die or laser cutting and then subsequent folding to create the kit 10, 100. Other ways to form the container box “blank” are envisioned herein.

It should be noted that the supply kit 10, 100 of the present invention is configured and sized with variable proportions, including, but not limited to proprietary labels, artwork, design, and folding parameters, inclusive of existing perforations and malleable creases. Also, the supply kit 10, 100 of the present invention can be re-scaled and/or be resized in accordance with the design and methodology of the present invention.

It would be appreciated by those skilled in the art that various changes and modifications can be made to the illustrated embodiments without departing from the spirit of the present invention. All such modifications and changes are intended to be covered by the appended claims.

What is claimed is:

1. A multi-purpose medical supply kit, comprising:
a sheet of material that is foldable into a first configuration as a storage box with a closable lid thereby defining a chamber therein; medical supplies being storable in the chamber;
a port in a wall of the storage box configured to receive used, non-sterile needles therethrough for storage in the chamber; and
the storage box being reconfigurable into a laptop stand.

2. The supply kit of claim 1, wherein the port is located through a rear panel of the storage box.

3. The supply kit of claim 2, wherein the port is a partially perforated pass through port providing a door flap.

4. The supply kit of claim 1, wherein the port is located through a front panel of the storage box.

5. The supply kit of claim 4, wherein the port is a fully perforated pass through port with a fully removable door panel thereby providing an open port.

6. The supply kit of claim 4, wherein the port is configured and arranged to be covered by a front flap of the storage box.

7. The supply kit of claim 6, further comprising adhesive on an interior surface of the front flap to seal the port.

8. The supply kit of claim 1, further comprising a hanger cord attached to the storage box.

9. The supply kit of claim 1, wherein the sheet of material is foldable and portions thereof are secured to each other with a releasable fastener.

10. The supply kit of claim 9, wherein the releasable fastener is hook and loop fastening material or adhesive.

11. A multi-purpose medical supply kit, comprising:
a sheet of material that is foldable into a first configuration as a storage box with a closable lid thereby defining a chamber therein; medical supplies being storable in the chamber;
a port in a wall of the storage box configured to receive used needles therethrough for storage in the chamber; and
the storage box being reconfigurable into a needle collection vessel.

12. A method of converting a medical supply kit into a laptop stand, comprising the steps of:
providing a sheet of material that is folded into a first configuration as a storage box having a container base with a bottom surface and with a top edge and a closable lid with a front flap and side flaps; the closable lid being hingedly connected to the base and defining a chamber therein;
opening the lid and releasing the side flaps and front flaps from each other;
reversing the front flap and the side flaps 180 degrees;
securing opposing edges of the front flap to the side flaps to provide a laptop base;
resting the base on the top edge of the container base; and
inverting the laptop base and container base with the front flap and side flaps resting on a support surface with bottom surface of the base facing upwards to provide a support surface for a laptop.

13. The method of claim 12, further comprising the step of:
providing a partially perforated needle receiving opening in a rear panel of the storage box.

14. The method of claim 12, further comprising the step of:
providing a fully perforated needle receiving opening in a front panel of the storage box.
